SEQUENCE LISTING (94 sequences)

SEQ ID NO: 1 - protein, 20 aa, Artificial
  Oligopeptide
RQQWELQGDRRCQSQLERAN

SEQ ID NO: 2 - protein, 20 aa, artificial
  Oligopeptide
WELQGDRRCQSQLERANLRP

SEQ ID NO: 3 - protein, 20 aa, Artificial
  Oligopeptide
QGDRRCQSQLERANLRPCEQ

SEQ ID NO: 4 - protein, 20 aa, Artificial
  Oligopeptide
RRCQSQLERANLRPCEQHLM

SEQ ID NO: 5 - protein, 20 aa, Artificial Sequence
  Oligopeptide
QSQLERANLRPCEQHLMQKI

SEQ ID NO: 6 - protein, 20 aa, Artificial
  Oligopeptide
LERANLRPCEQHLMQKIQRD

SEQ ID NO: 7 - protein, 20 aa, Artificial
  Oligopeptide
ANLRPCEQHLMQKIQRDEDS

SEQ ID NO: 8 - protein, 20 aa, Artificial
  Oligopeptide
ANLRPCEQHLMQKIQRDEDS

SEQ ID NO: 9 - protein, 20 aa, Artificial
  Oligopeptide
RPCEQHLMQKIQRDEDSYER

SEQ ID NO: 10 - protein, 20 aa, Artificial
  Oligopeptide
LMQKIQRDEDSYERDPYSPS

SEQ ID NO: 11 - protein, 20 aa, artificial
  Oligopeptide
KIQRDEDSYERDPYSPSQDP

SEQ ID NO: 12 - protein, 20 aa, Artificial
  Oligopeptide
RDEDSYERDPYSPSQDPYSP

SEQ ID NO: 13 - protein, 20 aa, Artificial
  Oligopeptide
DSYERDPYSPSQDPYSPSQD

SEQ ID NO: 14 - protein, 20 aa, Artificial
  Oligopeptide
ERDPYSPSQDPYSPSQDPDR

SEQ ID NO: 15 - protein, 20 aa, Artificial
  Oligopeptide
PYSPSQDPYSPSQDPDRRDP

SEQ ID NO: 16 - protein, 20 aa, Artificial
  Oligopeptide
PYSPSQDPYSPSQDPDRRDP

SEQ ID NO: 17 - protein, 20 aa, Artificial
  Oligopeptide
DPYSPSQDPDRRDPYSPSPY

SEQ ID NO: 18 - protein, 20 aa, Artificial
  Oligopeptide
DPYSPSQDPDRRDPYSPSPY

SEQ ID NO: 19 - protein, 20 aa, Artificial
  Oligopeptide
PYDRRGAGSSQHQERCCNEL

SEQ ID NO: 20 - protein, 20 aa, Artificial
  Oligopeptide
RRGAGSSQHQERCCNELNEF

SEQ ID NO: 21 - protein, 20 aa, Artificial
  Oligopeptide
RRGAGSSQHQERCCNELNEF

SEQ ID NO: 22 - protein, 20 aa, Artificial
  Oligopeptide
SQHQERCCNELNEFENNQRC

SEQ ID NO: 23 - protein, 20 aa, Artificial
  Oligopeptide
QERCCNELNEFENNQRCMCE

SEQ ID NO: 24 - protein, 20 aa, Artificial
  Oligopeptide
CCNELNEFENNQRCMCEALQ

SEQ ID NO: 25 - protein, 20 aa, Artificial
  Oligopeptide
ELNEFENNQRCMCEALQQIM

SEQ ID NO: 26 - protein, 20 aa, Artificial
  Oligopeptide
EFENNQRCMCEALQQIMENQ

SEQ ID NO: 27 - protein, 20 aa, Artificial
  Oligopeptide
NNQRCMCEALQQIMENQSDR

SEQ ID NO: 28 - protein, 20 aa, Artificial
  Oligopeptide
NNQRCMCEALQQIMENQSDR

SEQ ID NO: 29 - protein, 20 aa, Artificial
  Oligopeptide
CEALQQIMENQSDRLQGRQQ

SEQ ID NO: 30 - protein, 20 aa, Artificial
  Oligopeptide
LQQIMENQSDRLQGRQQEQQ

SEQ ID NO: 31 - protein, 20 aa, Artificial
  Oligopeptide
IMENQSDRLQGRQQEQQFKR

SEQ ID NO: 32 - protein, 20 aa, Artificial
  Oligopeptide
NQSDRLQGRQQEQQFKRELR

SEQ ID NO: 33 - protein, 20 aa, Artificial
  Oligopeptide
DRLQGRQQEQQFKRELRNLP

SEQ ID NO: 34 - protein, 20 aa, Artificial
  Oligopeptide
QGRQQEQQFKRELRNLPQQC

SEQ ID NO: 35 - protein, 20 aa, Artificial
  Oligopeptide
QQEQQFKRELRNLPQQCGLR

SEQ ID NO: 36 - protein, 20 aa, Artificial
  Oligopeptide
QQFKRELRNLPQQCGLRAPQ

SEQ ID NO: 37 - protein, 20 aa, Artificial
  Oligopeptide
KRELRNLPQQCGLRAPQRCD

SEQ ID NO: 38 - protein, 20 aa, Artificial
  Oligopeptide
LRNLPQQCGLRAPQRCDLDV

SEQ ID NO: 39 - protein, 20 aa, Artificial
  Oligopeptide
LPQQCGLRAPQRCDLDVDSG

SEQ ID NO: 40 - protein, 20 aa, Artificial
  Oligopeptide
QCGLRAPQRCDLDVDSGGRD

SEQ ID NO: 41 - protein, 19 aa, Artificial
  Oligopeptide
LRAPQRCDLDVESGGRDRY

SEQ ID NO: 42 - protein, 20 aa, Artificial
  Oligopeptide
MRRERGRQGDSSSCERQVDR

SEQ ID NO: 43 - protein, 20 aa, Artificial
  Oligopeptide
ERGRQGDSSSCERQVDRVNL

SEQ ID NO: 44 - protein, 20 aa, Artificial
  Oligopeptide
RQGDSSSCERQVDRVNLKPC

SEQ ID NO: 45 - protein, 20 aa, Artificial
  Oligopeptide
DSSSCERQVDRVNLKPCEQH

SEQ ID NO: 46 - protein, 20 aa, Artificial
  Oligopeptide
SCERQVDRVNLKPCEQHIMQ

SEQ ID NO: 47 - protein, 20 aa, Artificial
  Oligopeptide
RQVDRVNLKPCEQHIMQRIM

SEQ ID NO: 48 - protein, 20 aa, Artificial
  Oligopeptide
DRVNLKPCEQHIMQRIMGEQ

SEQ ID NO: 49 - protein, 20 aa, Artificial
  Oligopeptide
NLKPCEQHIMQRIMGEQEQY

SEQ ID NO: 50 - protein, 20 aa, Artificial
  Oligopeptide
PCEQHIMQRIMGEQEQYDSY

SEQ ID NO: 51 - protein, 20 aa, Artificial
  Oligopeptide
QHIMQRIMGEQEQYDSYDIR

SEQ ID NO: 52 - protein, 20 aa, Artificial
  Oligopeptide
MQRIMGEQEQYDSYDIRSTR

SEQ ID NO: 53 - protein, 20 aa, Artificial
  Oligopeptide
IMGEQEQYDSYDIRSTRSSD

SEQ ID NO: 54 - protein, 20 aa, Artificial
  Oligopeptide
EQEQYDSYDIRSTRSSDQQQ

SEQ ID NO: 55 - protein, 20 aa, Artificial
  Oligopeptide
QYDSYDIRSTRSSDQQQRCC

SEQ ID NO: 56 - protein, 20 aa, Artificial
  Oligopeptide
SYDIRSTRSSDQQQRCCDEL

SEQ ID NO: 57 - protein, 20 aa, Artificial
  Oligopeptide
IRSTRSSDQQQRCCDELNEM

SEQ ID NO: 58 - protein, 20 aa, Artificial
  Oligopeptide
TRSSDQQQRCCDELNEMENT

SEQ ID NO: 59 - protein, 20 aa, Artificial
  Oligopeptide
SDQQQRCCDELNEMENTQRC

SEQ ID NO: 60 - protein, 20 aa, Artificial
  Oligopeptide
QQRCCDELNEMENTQRCMCE

SEQ ID NO: 61 - protein, 20 aa, Artificial
  Oligopeptide
CCDELNEMENTQRCMCEALQ

SEQ ID NO: 62 - protein, 20 aa, Artificial
  Oligopeptide
ELNEMENTQRCMCEALQQIM

SEQ ID NO: 63 - protein, 20 aa, Artificial
  Oligopeptide
EMENTQRCMCEALQQIMENQ

SEQ ID NO: 64 - protein, 20 aa, Artificial
  Oligopeptide
NTQRCMCEALQQIMENQCDR

SEQ ID NO: 65 - protein, 20 aa, Artificial
  Oligopeptide
RCMCEALQQIMENQCDRLQD

SEQ ID NO: 66 - protein, 20 aa, Artificial
  Oligopeptide
CEALQQIMENQCDRLQDRQM

SEQ ID NO: 67 - protein, 20 aa, Artificial
  Oligopeptide
LQQIMENQCDRLQDRQMVQQ

SEQ ID NO: 68 - protein, 20 aa, Artificial
  Oligopeptide
IMENQCDRLQDRQMVQQFKR

SEQ ID NO: 69 - protein, 20 aa, Artificial
  Oligopeptide
NQCDRLQDRQMVQQFKRELM

SEQ ID NO: 70 - protein, 20 aa, Artificial
  Oligopeptide
DRLQDRQMVQQFKRELMNLP

SEQ ID NO: 71 - protein, 20 aa, Artificial
  Oligopeptide
QDRQMVQQFKRELMNLPQQC

SEQ ID NO: 72 - protein, 20 aa, Artificial
  Oligopeptide
QMVQQFKRELMNLPQQCNFR

SEQ ID NO: 73 - protein, 20 aa, Artificial
  Oligopeptide
QQFKRELMNLPQQCNFRAPQ

SEQ ID NO: 74 - protein, 20 aa, Artificial
  Oligopeptide
KRELMNLPQQCNFRAPQRCD

SEQ ID NO: 75 - protein, 20 aa, Artificial
  Oligopeptide
LMNLPQQCNFRAPQRCDLDV

SEQ ID NO: 76 - protein, 20 aa, Artificial
  Oligopeptide
LPQQCNFRAPQRCDLDVSGG

SEQ ID NO: 77 - protein, 19 aa, Artificial
  Oligopeptide
QCNFRAPQRCDLDVSGGRC

SEQ ID NO: 78 - protein, 149 aa, Peanut
TILVALALFLLAAHASARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVE

SEQ ID NO: 79 - protein, 121 aa, Peanut
MRRERGRGGDSSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVS

SEQ ID NO: 80 - protein, 9 aa, Artificial
  Oligonucleotide
RRCQSQLER

SEQ ID NO: 81 - protein, 11 aa, Artificial
  Oligonucleotide
RPCEQHLMQKI

SEQ ID NO: 82 - protein, 6 aa, Artificial
  Oligopeptide
DPYSPS

SEQ ID NO: 83 - protein, 10 aa, Artificial
  Oligopeptide
CNELNEFENN

SEQ ID NO: 84 - protein, 13 aa, Artificial
  Oligopeptide
CEALQQIMENQSD

SEQ ID NO: 85 - protein, 6 aa, Artificial
  Oligopeptide
LQGRQQ

SEQ ID NO: 86 - protein, 8 aa, Artificial
  Oligopeptide
KRELRNLP

SEQ ID NO: 87 - protein, 13 aa, Artificial
  Oligopeptide
CGLRAPQRCDLDV

SEQ ID NO: 88 - protein, 14 aa, Artificial
  Oligopeptide
MRRERGRGGDSSSS

SEQ ID NO: 89 - protein, 11 aa, Artificial
  Oligopeptide
KPCEQHIMQRI

SEQ ID NO: 90 - protein, 6 aa, Artificial
  Oligopeptide
YDSYDI

SEQ ID NO: 91 - protein, 10 aa, Artificial
  Oligopeptide
CDELNEMENT

SEQ ID NO: 92 - protein, 13 aa, Artificial
  Oligopeptide
CEALQQIMENQCD

SEQ ID NO: 93 - protein, 8 aa, Artificial
  Oligopeptide
KRELMNLP

SEQ ID NO: 94 - protein, 13 aa, Artificial
  Oligopeptide
CNFRAPQRCDLDV

(19) United States
(12) Patent Application Publication
(10) Pub. No.: US 2014/0080730 A1
(43) Pub. Date: Mar. 20, 2014

(54) METHOD FOR PREDICTING SEVERITY OF ALLERGIC REACTION

(71) Applicants: Stephen C. Dreskin, Denver, CO (US); Kanao Otsu, Denver, CO (US)

(72) Inventors: Stephen C. Dreskin, Denver, CO (US); Kanao Otsu, Denver, CO (US)

(73) Assignee: The Regents of the University of Colorado, a body corporate, Denver, CO (US)

(21) Appl. No.: 14/006,448

(22) PCT Filed: Mar. 20, 2012
(86) PCT No.: PCT/US12/29842

§ 371 Date: Dec. 2, 2013

Related U.S. Application Data
(60) Provisional application No. 61/454,538, filed on Mar. 20, 2011.

(51) Int. Cl. G01N 33/68

(52) U.S. Cl. CPC G01N 33/6854
USPC 506/9; 436/501

(57) ABSTRACT

The present invention provides methods for determining a likelihood of a degree of allergic reaction or a severity thereof using a threshold value rather than the binding activity number between the immunoglobulin of the subject and a test composition comprising a marker protein moiety of the allergy inducing material. In one particular embodiment of the invention, methods are provided for determining a likelihood of a degree of allergic reaction severity of a subject to peanuts using a threshold value of binding between immunoglobulin E (IgE) of the subject and Ara h 2 moiety and Ara h 6 moiety.

### CROSS-REFERENCE TO RELATED APPLICATIONS

This application claims the priority benefit of U.S. Provisional Application No. 61/454,538, filed Mar. 20, 2011, which is incorporated herein by reference in its entirety.

### STATEMENT REGARDING FEDERALLY FUNDED RESEARCH

This invention was made with government support under grant number AI052164 awarded by the National Institutes of Health. The government has certain rights in the invention.

### FIELD OF THE INVENTION

The present invention relates to methods for determining a likelihood of a degree of allergic reaction or a severity thereof using a threshold value rather than the binding activity number between the immunoglobulin of the subject and a test composition comprising a marker protein moiety of the allergy inducing material. In one particular embodiment of the invention, methods are provided for determining a likelihood of a degree of allergic reaction severity of a subject to peanuts using a threshold value of binding between immunoglobulin E (IgE) of the subject and Ara h 2 moiety and Ara h 6 moiety.

### BACKGROUND OF THE INVENTION

Conventional methods for determining the likelihood of severity of allergic reaction in a subject to a particular allergen use the binding activity (e.g., degree of binding associated with a 95% confidence of any reaction) of a particular immunoglobulin in the subject and the known allergy inducing protein or material of the allergy inducing material such as peanuts, milk, eggs, weeds and other plants, animals, etc.

While the binding activity maybe useful in identifying whether a subject is allergic to a particular material, it does not provide a significantly useful information on the degree of severity of potential allergic reaction to the material by the subject. For example, it has been shown that some people who are allergic to peanuts have shown a high binding activity (e.g., units/ml) of subject's IgE to peanut protein such as Ara h 2, but they do not show a potentially life threatening allergic reaction. Conversely, it has been shown that some people who are allergic to peanuts have shown a relatively low binding activity between the subject's IgE and peanut protein Ara h 2, but their allergic reaction to peanuts is potentially life threatening.

Accordingly, currently there is no method for relatively accurately predicting severity of a subject's allergic reaction to a particular allergen. Therefore, there is a need for methods to accurately predict or determine the likelihood of degree of severity of allergic reaction to a particular allergen in subjects.

### SUMMARY OF THE INVENTION

Some aspects of the invention provide methods for identifying a therapeutically useful compound for treating peanut allergy in a subject. Such methods typically comprises determining the level of inhibitory activity of a compound against binding of Immunoglobulin E (IgE) of a subject allergic to peanuts to a molecule comprising amino acid sequence of Ara h 2 peptide, Ara h 6 peptide, or an epitope thereof, or an isoform thereof, or a combination thereof. It should be appreciated that one skilled in the art can readily determine the level of inhibitory activity that is useful in such methods, for example, by balancing the type and/or severity of side-effect versus the efficacy in treating peanut allergy. One particular example level of inhibitory activity that is useful in treating peanut allergy is IC₅₀ of about 100 μM or less, typically about 50 μM or less, and often about 10 μM or less. However, it should be appreciated that the scope of the invention is not limited to these particular IC₅₀ values.

Other aspects of the invention provide methods for predicting severity of allergic reaction to peanuts in a subject, said method comprising determining the level of binding activity of the subject's immunoglobulin E (IgE) to a molecule comprising an amino acid sequence of Ara h 2 peptide, Ara h 6 peptide, or an epitope thereof, or an isoform thereof, or a combination thereof. Typically, the level of binding activity of the subject's IgE to the amino acid sequence is indicative of the subject's severity of allergic reaction of the subject to peanuts.

Still other aspects of the invention provide methods for predicting the likelihood of successful desensitization to allergic reaction to peanuts in a subject. Such methods generally comprise determining the level of binding activity or a threshold value of the subject's immunoglobulin E (IgE) to a molecule comprising an amino acid sequence of Ara h 2 protein moiety, Ara h 6 protein moiety, or an epitope thereof, or an isoform thereof, or a combination thereof. Often the level of binding activity or the threshold value of the subject's IgE to the amino acid sequence is indicative of the likelihood of successful desensitization therapy to peanut allergic reaction of the subject.

In some embodiments, the amino acid sequence comprises amino acid sequence of an epitope of Ara h 2 protein moiety, Ara h 6 protein moiety, or an isoform thereof, or a combination thereof. In some particular embodiments, the amino acid sequence of the epitope of Ara h 2 protein moiety comprises an amino acid sequence of SEQ ID NOS:80-87; or an isoform thereof; or a combination thereof. Yet in other particular embodiments, the amino acid sequence of the epitope of Ara h 6 protein moiety comprises an amino acid sequence of SEQ ID NOS:88-94; or an isoform thereof; or a combination thereof.

Typically, methods of the invention use a plurality of amino acid sequences of the epitope(s) of Ara h 2 protein moiety (e.g., SEQ ID NOS:1-41 and/or 80-87) and a plurality of amino acid sequences of the epitope(s) of Ara h 6 protein moiety (e.g., SEQ ID NOS:42-77 and/or 88-94) are used in combination. In some embodiments, at least five, typically at least eight, often at least fifteen, and more often at least thirty Ara h 2 protein moieties are used in methods of the invention. Within these embodiments, in general any combination of SEQ ID NOS:1-41 and 80-87 is used. In other embodiments, methods of the invention use at least five, typically at least seven, often at least fifteen, and more often at least thirty Ara h 6 protein moieties. Within these embodiments, in general any combination of SEQ ID NOS:42-77 and 88-94 is used.

Still further, combinations different embodiments of the number of Ara h 2 and Ara h 6 protein moieties described herein form other embodiments. For example, in one particularly embodiment, methods of the invention use at least eight Ara h 2 protein moieties selected from SEQ ID NOS:1-41 and 80-87, and at least seven Ara h 6 protein moieties selected from SEQ ID NOS:42-77 and 88-94. In this manner, a variety of embodiments are encompassed within the scope of the invention.

It should be appreciated that while the scope of the invention includes any and all possible combinations of such SEQ ID NOS., each individual combination is not listed herein for the sake of brevity.

### BRIEF DESCRIPTION OF THE DRAWINGS

FIG. 1 is a table of peptides of Ara h 2 (1-41) (SEQ ID NOS:1-41, respectively) and Ara h 6 (1-36) (SEQ ID NOS:42-77, respectively) that were used in microarray assay to determine the severity of peanut allergy.

FIGS. 2A and 2B are graph showing the percent of subjects binding to each of 41 peptides for Ara h 2 and to each of 36 peptides for Ara h 6, respectively.

FIGS. 3A-3D are representative graphs of IgE binding (z-scores) to peptides of Ara h 2 and Ara h 6 according to grades of reported clinical reactions following natural exposure to peanuts. A horizontal lines is placed at a z-score=3.

FIG. 4 shows amino acid alignment for Ara h 2 (SEQ ID NO:78) and Ara h 6 (SEQ ID NO:79) (Top). Different colors denote epitopes for Ara h 2 (SEQ ID NOS:80-87) and Ara h 6 (SEQ ID NOS:88-94) and these are listed on the lower half of FIG. 4.

FIG. 5 is heat maps of IgE-binding to individual peptides (z-scores) for peptides of Ara h 2 (left panel) and Ara h 6 (right panel). The subjects were first sorted according to their grades of reaction obtained by history. The range of z-scores is (−2 to ≧5).

FIGS. 6A and 6B are heat maps of unsupervised cluster analysis of the data from FIG. 5 for Ara h 2 and Ara h 6, respectively. Sera from subjects with a history of Grade III or IV reactivity are in red.

FIG. 7 is heat maps of binary (yes, z>3 or no, z≦3) binding of IgE to peptides for Ara h 2 (left panel) and Ara h 6 (right panel) for all 30 sera. The subjects were first sorted according to their grades of reaction obtained by history. ‘Yes’ is denoted in yellow and ‘No’ is denoted in blue.

FIGS. 8A and 8B are heat maps of unsupervised cluster map of the data from FIG. 7 for Ara h 2 and Ara h 6, respectively. ‘Yes’ is denoted in yellow and ‘No’ is denoted in blue. Sera from subjects with a history of Grade III or IV reactivity are in red.

FIG. 9 is heat maps of binary (yes/no; z≦3/z>3) binding of IgE to epitopes for Ara h 2 (left panel) and Ara h 6 (right panel) for all 30 sera sorted according to grades of reaction based on clinical history. ‘Yes’ is denoted in yellow and ‘No’ is denoted in blue.

FIGS. 10A and 10B are unsupervised cluster analysis of the data from FIG. 9 for Ara h 2 and Ara h 6, respectively. ‘Yes’ is denoted in yellow and ‘No’ is denoted in blue. Sera from subjects with a history of Grade III or IV reactivity are in red.

FIGS. 11A-11D are contingency plots of key data from the unsupervised cluster analysis shown in FIGS. 10A and 10B. Totals for each horizontal and vertical set of numbers are shown. For all plots, n=30. A) Subjects with a history of grades I/II severity cluster in either node 1 or 2A of Ara h 2 (p<0.03); B) Subjects with a history of grades III/IV severity cluster in node 1B3B of Ara h 6 (p<0.03); C) Subjects who cluster in node 1B3B of Ara h 6 do not cluster in either node 1 or 2A of Ara h 2 (p=0.0015); D) Subjects who have the “high risk pattern” (h 6 (1B3B), yes and h 2 (1+2A), no) predominantly have a history of grades III/IV severity (OR=12.6; 95% CI=2.0, 79.5; p<0.007).

### DETAILED DESCRIPTION OF THE INVENTION

### Definitions

As used herein, the term “threshold value” refers to a statistically derived value in which the binding between a ligand and the receptor (e.g., binding between IgE and Ara h 2, IgE and Ara h 6, etc.) is a statistically derived value where the p-value is 0.01 or less, typically 0.005 or less, and often 0.003 or less. For a discussion on the threshold value see Lin et al., in J Allergy Clin Immunol, 2009, 124, 315-22, 22 e1-3.

When the term “moiety” is used in conjunction with a name of a peptide, the term includes the peptide itself, an epitope thereof, an isoform thereof, an oligopeptide comprising the epitope thereof, an oligopeptide comprising an isoform thereof, an oligopeptide comprising at least 75%, typically at least 80%, often at least 90%, and more often at least 95% amino acid sequence homologue of the peptide, an oligopeptide comprising at least 75%, typically at least 80%, often at least 90%, and more often at least 95% of amino acid sequence of an epitope of the peptide, and a combination thereof. For example, the term “Ara h 2 moiety” refers to Ara h 2 peptide, an epitope of Ara h 2, an isoform of Ara h 2, an oligopeptide comprising the epitope of Ara h 2, an oligopeptide comprising an isoform of Ara h 2, an oligopeptide comprising at least 75%, typically at least 80%, often at least 90%, and more often at least 95% amino acid sequence homologue of Ara h 2, an oligopeptide comprising at least 75%, typically at least 80%, often at least 90%, and more often at least 95% of amino acid sequence of an epitope of Ara h 2, and a combination thereof. Similarly, the term “Ara h 6 moiety” refers to Ara h 6 peptide, an epitope of Ara h 6, an isoform of Ara h 6, an oligopeptide comprising the epitope of Ara h 6, an oligopeptide comprising an isoform of Ara h 6, an oligopeptide comprising at least 75%, typically at least 80%, often at least 90%, and more often at least 95% amino acid sequence homologue of Ara h 6, an oligopeptide comprising at least 75%, typically at least 80%, often at least 90%, and more often at least 95% of amino acid sequence of an epitope of Ara h 6, and a combination thereof.

The term “allergy inducing material” refers to a material that is known to cause an allergic reaction in a subject. Exemplary allergy inducing materials include, but are not limited to, food, such as peanuts, eggs, milk, shellfish (e.g., shrimp, clams, scallops, lobster), etc.; plant pollens, from such as trees, grasses, weeds, flowers, etc.; animal danders and epithelium, such as from cats, dogs, mice, rats, guinea pigs; and other environmental agents such as dust mites, feathers, and cockroaches, etc.

The term “subject” refers to an animal, typically a mammal such as human, primates, cats, dogs, etc., and often human.

### Methods of the Invention

Some aspects of the invention provide methods for predicting or determining a likelihood of degree of severity of allergic reaction to an allergy inducing material in a subject. Unlike conventional methods that analyze the binding activity (e.g., IC₅₀ value) of an immunoglobulin and a marker protein moiety of the allergy inducing material, methods of the invention utilize a threshold value to predict and/or determine the likelihood of the degree of severity of allergic reaction to an allergy inducing material. It has been found by the present inventors that utilizing the binding activity is not an accurate predictor of the severity or degree of allergic reaction in a subject. For example, some subject having a relatively large binding activity have shown to possess a non-life-threatening allergic reaction to a given allergy inducing material. And conversely, some subjects having a relatively low binding activity have been shown to posses a severe and often life-threatening allergic reaction to a given allergy inducing material. Such inconsistent results based on binding activity have led the present inventors to discover methods of the invention which use a threshold value. Methods of the invention have shown to be a significantly better predictor or a significantly better at determining the likelihood of a subject's degree or severity of allergic reaction to an allergy inducing material.

Methods of the invention include contacting a fluid sample of the subject with a test composition comprising a marker protein moiety of the allergy inducing material, and analyzing the binding between the marker protein moiety of the allergy inducing material and the immunoglobulin in the fluid sample of the subject to predict or determine the subject's likelihood of degree of severity of allergic reaction to the allergy inducing material. The marker protein moieties are typically chosen based on evidence or experimental data that the allergic effector activity of the marker protein moieties comprise a plurality of the total allergic effector activity of the allergy inducing material. Allergenic activity of marker proteins is often determined in a functional assay, e.g., assays that are RBL-based, basophil-based, mast cell based, etc.

The fluid sample of the subject comprises an immunoglobulin of the subject that is known or suspected to cause an immune response in the presence of the marker protein in a control group that is allergic to the allergy inducing material. It should be appreciated that the immunoglobulin of a particular subject may not cause an immune response. However, the immunoglobulin that is present in the fluid sample of the subject is known to cause immune response in subjects that are allergic to the allergy inducing material.

The marker protein moiety includes a protein moiety or at least a portion of the allergy inducing/causing protein moiety that is known to cause an immune response in the control group that is allergic to the allergy inducing material. Such a protein moiety can be the entire protein itself or a composition that comprises the entire protein (e.g., with a linker to a solid support), an epitope of marker protein, an isoform of marker protein, an oligopeptide comprising the epitope of marker protein, an oligopeptide comprising an isoform of marker protein epitope, an oligopeptide comprising at least 75%, typically at least 80%, often at least 90%, and more often at least 95% amino acid sequence homologue of the marker protein, an oligopeptide comprising at least 75%, typically at least 80%, often at least 90%, and more often at least 95% of amino acid sequence of an epitope of the protein, and/or a combination thereof.

The fluid sample of the subject can be any fluid obtained from the subject that includes the immunoglobulin of the subject that is known or suspected to cause an immune response in the presence of the marker protein in a control group that is allergic to the allergy inducing material. Exemplary fluid samples that are useful in methods of the invention include, but are not limited to, blood, serum, plasma, and body secretions such as nasal secretions and fluid obtained from the lung (e.g., bronchoalveolar lavage fluid), and a combination thereof.

Analysis of the binding between the marker protein moiety of the allergy inducing material and the immunoglobulin in the fluid sample of the subject includes analyzing whether the binding activity is above and/or below a threshold value. The threshold value is determined by analyzing a the binding activity from a control group and is typically a preset threshold value that is determined experimentally. It should be appreciated that as more data is gathered and analyzed the threshold value may change. The threshold value need not be determined by the one practicing the method of the invention, it can be a published value by others. Typically, the threshold value is obtained by analyzing statistically significant amount of data sample from a control group. An exemplary method for determining a threshold value is illustrated in the Examples section infra.

In some embodiments of the invention, the test composition comprises a plurality of marker protein moieties of the allergy inducing material. Generally, as the number of marker protein moieties analyzed increases, the accuracy of predicting or determining the likelihood of allergic reaction severity increases. However, the cost of analysis generally also increases as the number of marker protein moieties analyzed increases. Typically, methods of the invention provide at least 80%, often at least 85%, more often at least 90%, and most often at least 95% accuracy in predicting or determining the likelihood of allergic reaction severity in a subject to a particular allergy inducing material.

In other embodiments, the test composition comprises a microarray. For example, the marker protein moiety can be affixed to a microarray for a fast and simultaneously multiple analysis. Use of microarrays in determining binding between a ligand and a receptor (e.g., between an immunoglobulin and a marker protein moiety) is generally well known to one skilled in the art. However, what is not known prior to the discovery by the present inventors is the use of a threshold value in determining the severity of allergic reaction in a subject to a particular allergy inducing material.

Yet in other embodiments, analyzing the value of binding between the marker protein moiety of the allergy inducing material and the immunoglobulin in the fluid sample of the subject includes converting the threshold value to an optically analyzable pattern; and comparing the optically analyzable pattern to a reference pattern. As with determining the threshold value, one need not prepare the reference pattern by the one practicing the method of the invention, the reference pattern can be published or provided by others as long as the method of converting the threshold value to an optically analyzable pattern is consistent.

Still in some embodiments, the reference pattern comprises an optically analyzable pattern of binding at or above threshold binding activity value between the marker protein moiety of the allergy inducing material and the immunoglobulin in the fluid sample of the control subject that is allergic to the marker protein moiety of the allergy inducing material. It should be appreciated that there can be a multiple reference patterns, e.g., patterns for each degree of allergic reaction severity. Accordingly, one can have or provide a multiple levels or degree or severity of allergic reaction to a given allergy inducing material.

It should be appreciated that the scope of the invention also includes a “negative control.” That is, the reference pattern can comprise an optically analyzable pattern of binding above threshold between the marker protein moiety of the allergy inducing material and the immunoglobulin in the fluid sample of the control subject that is less allergic or not allergic to the marker protein moiety of the allergy inducing material.

The reference pattern can also comprise an optically analyzable pattern of threshold value of binding between the marker protein moiety of the allergy inducing material and the immunoglobulin in the fluid sample of the control subject that is substantially not allergic to the marker protein moiety of the allergy inducing material.

Methods of the invention can also be used to develop therapeutically useful molecules (including, but not limited to, peptides, small molecules, etc.) to alleviate or even prevent allergic reaction to an allergy inducing material. For example, one can use the methods of the invention as molecular assays to screen and identify potentially useful molecules in treating or preventing allergic reaction to a particular allergy inducing material.

Other aspects of the invention include normalizing the fluid sample concentration of the subject based on values from previous allergy tests prior to said step of contacting the fluid sample of the subject with the test composition.

One particular aspect of the invention provides methods for predicting or determining a likelihood of a degree of peanut allergy severity in a subject. Currently there is no diagnostic testing to predict the severity of the peanut allergy reaction in a subject. Peanuts are a major cause of severe immunoglobulin E (IgE) mediated food allergy affecting approximately 1.4% of the population in the United States. Allergy to peanuts rarely resolves in an allergic individual and remains a source of morbidity in approximately 80% of peanut-allergic patients. Although levels of anti-peanut IgE >14 kU/ml and skin tests of >5 mm are associated with a positive oral peanut challenge and binding of IgE to Ara h 2 is associated with clinical reactivity to peanuts, there are no in vitro tests that correlate with severity of clinical history or responses to food challenges.

Currently, eleven peanut allergens (Ara h 1-11) have been identified. Patients with a more severe clinical history are reported to have IgE that recognizes a greater number of linear epitopes of Ara h 1, 2 and 3. Of eleven peanut allergens identified to date, the 2S albumins, Ara h 2 and Ara h 6, account for approximately 10% of the peanut proteome. It has also been found that Ara h 2 and Ara h 6 proteins (SEQ ID NOS:78 and 79, respectively) share about 60% sequence homology (see FIG. 4), are highly homologous in their secondary structure (4 and 5 disulfide bonds respectively), and are the most potent peanut allergens. Studies have also shown that Ara h 2 and Ara h 6 together account for 78±4% of the total effector activity in crude peanut extracts (CPE) in most patients with peanut allergy who have been examined to date. Ara h 2 and Ara h 6 share 59% homology in their primary amino acid sequence. Data from the present inventors' study indicate that there is great redundancy in the ability of Ara h 2 and Ara h 6 to cross-link IgE.

Specific antibody-peptide interactions have been mapped using a microarray immunoassay technology. In fact, microarray immunoassays with overlapping peptides that bind IgE have been described for a variety of food allergens. For peanut allergy, this mapping study resulted in defining 9 core epitopes of Ara h 2 ranging from 6-16 residues in length 78% of which was consistent with previous mapping using SPOT method.

Given the structural homology between Ara h 2 and Ara h 6 and their potent effector activity, the present inventors have developed a microarray assay with Ara h 2 and Ara h 6 peptides to examine IgE binding to linear epitopes of these proteins. It should be noted that Ara h 6 has not been previously studied in this fashion. This assay revealed subtleties regarding IgE binding to linear epitopes of these two related proteins and a surprising and unexpected correspondence of these finding to the severity of clinical histories. Methods of the invention have a wide variety of clinical utilities including identifying subjects at risk for more severe clinical allergic reactions to peanuts and screening for or identifying compounds or molecules that can alleviate or prevent allergic reaction in a subject to peanuts.

In one particular embodiment, methods for predicting or determining a likelihood of a degree of peanut allergy severity in a subject include contacting a fluid sample of the subject comprising immunoglobulin E (IgE) with a test composition comprising a marker protein moiety of peanut. The marker protein moiety comprises a mixture of: (i) Ara h 2 protein moiety; and (ii) Ara h 6 protein moiety. In some instances the marker protein moiety comprises a plurality Ara h 2 protein moieties, a plurality of Ara h 6 protein moieties, or a combination thereof. Such methods also include analyzing the value of binding between the marker protein of peanut and IgE in the fluid sample of the subject to determine the subject's likelihood of degree of severity of allergic reaction to peanut.

In some embodiments, the fluid sample of the subject comprises blood, serum, plasma, and body secretions such as nasal secretions and fluid obtained from the lung (bronchoalveolar lavage fluid), or a combination thereof.

Still in other embodiments, the test composition comprises a microarray.

Yet in other embodiments, the method of analyzing the binding value comprises converting the binding value to an optically analyzable pattern; and comparing the optically analyzable pattern to a reference pattern.

In some embodiments, the reference pattern comprises an optically analyzable pattern of binding threshold value between Ara h 2 protein moiety and Ara h 6 protein moiety of peanuts and IgE in the fluid sample of the control subject that is allergic to peanuts. It should be appreciated that the optically analyzable pattern for Ara h 2 protein moiety and Ara h 6 protein moiety can be compared separately or as a combination. Typically, the optically analyzable pattern for Ara h 2 protein moiety and the optically analyzable Ara h 6 protein moiety are compared separately.

In other embodiments, the reference pattern comprises an optically analyzable pattern of binding threshold value between Ara h 2 protein moiety and Ara h 6 protein moiety of peanuts and IgE in the fluid sample of the control subject that is substantially not allergic to peanuts.

Still in other embodiments, methods for predicting or determining a likelihood of a degree of peanut allergy severity in a subject further comprise normalizing the fluid sample concentration of the subject based on values from previous allergy tests prior to said step of contacting the fluid sample of the subject with the test composition. Typically, normalization involves adjusting the fluid sample (e.g., either by dilution or by concentration) such that the concentration of IgE is substantially similar to that used to generate the reference pattern.

Unlike conventional method of binding assay, methods of the invention typically include determining the concentration of the immunoglobulin (e.g., IgE and the food or other allergen-specific IgE) in the fluid sample, and adjusting the concentration of the immunoglobulin such as IgE (e.g, by dilution or concentration of the fluid sample) such that a substantially similar concentration of total, food, or other allergen specific-immunoglobulin is used in both the reference pattern and the fluid sample of the subject. As used herein, the term “substantially similar” or any numeric value provided herein includes a variation of ±10%, and often ±5% of the value. Thus, for example, IgE concentration of 20 μM refers to IgE concentration of 20 μM±2 μM, i.e., it includes a range of IgE concentration from 18 μM to 22 μM.

Given the structural homology between Ara h 2 and Ara h 6 and their potent effector activity, in some embodiments, methods of the invention utilize a microarray assay with Ara h 2 protein moiety and Ara h 6 protein moiety to examine IgE binding to linear epitopes of these proteins. In some instances, this assay revealed subtleties regarding IgE binding to linear epitopes of these two related proteins and a surprising and unexpected correspondence of these finding to the severity of clinical histories.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

This example illustrates one embodiment of the invention where a likelihood of a degree of peanut allergy severity in a subject can be predicted.

Abbreviations used: 20 kD fraction, gel filtration chromatography fraction containing Ara h 2 and Ara h 6; BE, binding events; CPE, Crude peanut extract; CPE recombined, CPE with 20 kD fraction; CPE recombined w/o 20 kD (also, CPE w/o 20 kD), CPE excluding the 20 kD fraction; PBE, possible binding events; TBE, total binding events.

Thirty highly peanut-allergic subjects were stratified by clinical history. IgE binding to overlapping 20-mer peptides of Ara h 2 and Ara h 6 was assessed using microarrays.

Each individual demonstrated a unique IgE-binding fingerprint to peptides and these data were coalesced into epitope binding. IgE from subjects with a history of more severe reactions (n=19) had a smaller frequency of binding events (BE) for both Ara h 2 (52 BE out of 152 possible binding events (PBE)) and Ara h 6 (13 BE out of 133 PBE) compared to IgE from those with milder histories (n=11) (Ara h 2: 48 BE out of 88 PBE, p<0.003; Ara h 6: 24 BE out of 77 PBE, p=0.001). Using an unsupervised hierarchal cluster analysis, subjects with similar histories tend to cluster and we have identified a “high risk pattern” of binding to peptides of Ara h 2 and Ara h 6 that is found predominantly in subjects with a history of more severe reactions (OR=13.5; 95% CI: 2.15-84.7; p<0.007).

IgE from patients with more severe clinical histories recognize fewer linear epitopes of both Ara h 2 and Ara h 6 and bind these epitopes in characteristic patterns. This may have prognostic value.

Subjects and classification: A total of 47 subjects, ages 7-70, with a strong history of peanut-induced immediate hypersensitivity reactions were enrolled for this study. The thirty subjects who participated in this study had peanut specific IgE >14 KU/L, (ImmunoCap®, Phadia; Uppsala, Sweden), a finding consistent with 95% confidence of true peanut allergy. The patients' reported symptoms following naturally occurring exposure to peanuts were classified into grades of anaphylaxis according to criteria established by the World Health Organization for evaluation of allergic reactions in the context of allergen-specific immunotherapy: grade I, cutaneous symptoms alone (angioedema, rash, hives); grade II, two-organ system involvement (e.g. lower respiratory and cutaneous, or gastrointestinal and lower respiratory); grade III, three or more organ system involvement and/or those with upper respiratory involvement (upper respiratory angioedema, stridor); grade IV, cardiovascular compromise (shock, decrease pulse, syncope) (Table A).

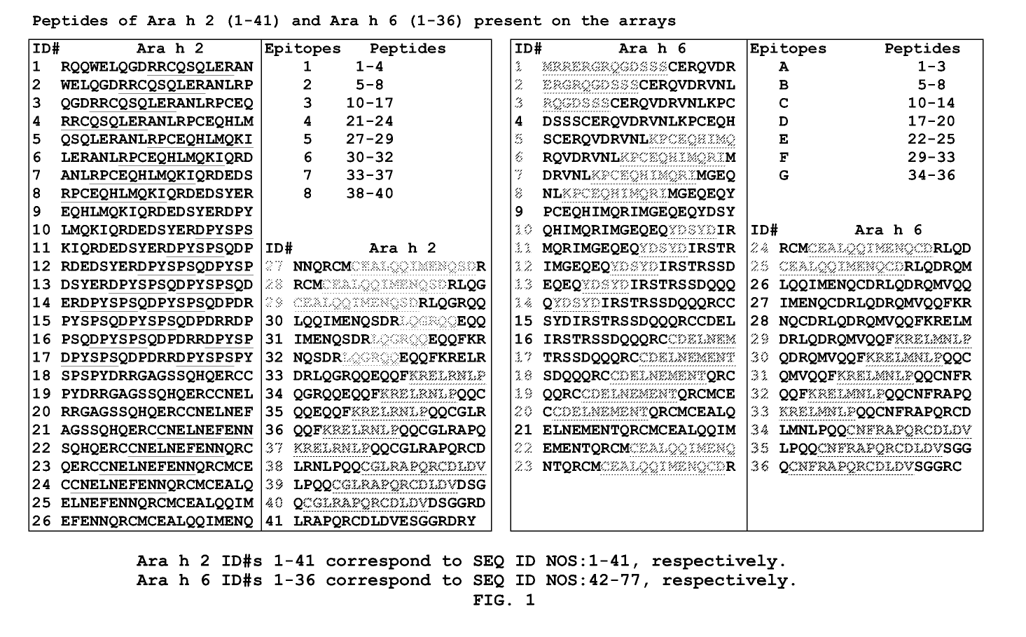

Peptides: A library of peptides, consisting of 20 amino acids (AAs) in length overlapping by 17 AAs (offset of 3) covering the entire sequence of Ara h 2 (SEQ ID NOS:1-41) and its isoforms, and Ara h 6 (SEQ ID NOS:42-77) and its isoforms were synthesized (JPT Peptide Technologies; Berlin, Germany) (FIG. 1). The PepStar™ peptide microarray platform, which places a 403 Dalton linker arm at the C-terminus of each peptide, was used to generate customized peptide microarrays on glass slides (JPT Peptide Technologies) as described by Shreffler et al. in J Allergy Clin Immunol, 2005, 116, 893-9. Purified Ara h 2, Ara h 6, human IgE, and empty spots were used as positive and negative controls.

Microarray printing: Triplicate spots were printed on APiX Protein Microarray Slides coated with a proprietary optically clear nitrocellulose (Gentel Biosciences; Madison, Wis.). All slides were stored at 4° C. and were used within 6 months of the manufactured date.

Immunoassay: Slides were placed in a SIMplex™ 64 well device (Gentel Biosciences), washed with 150 μL of PBS with 0.05% Tween 20 (PBST) with 1% BSA. In preliminary experiments, it was determined that the assay performed well with peanut-specific IgE values of 20-30 ng/ml (data not shown). In the experiments reported here, 70 μl of sera were diluted in PBST with 0.1% BSA (final concentration of peanut-specific IgE was 22 ng/ml), applied to each array and incubated overnight on a rocker at 4° C. Slides were washed, incubated for 60 minutes on a plate shaker at 160 rpm at room temperature with biotinylated murine monoclonal anti-human IgE (1:10,000) (Invitrogen; Carlsbad, Calif.). The slides were washed and developed with anti-biotin Gold Conjugate followed by SilverQuant® Reagents A and B, washed and dried using nitrogen gas (Gentel Biosciences).

### Analysis of Microarray Data

Peptides. The slides were scanned in an APiX Scanner (Gentel Biosciences) and peptide-specific and background signals were determined. Z-scores were calculated as described by Lin et al. (J Allergy Clin Immunol, 2009, 124, 315-22, 22 e1-3) with binding to an individual peptide considered to be positive if the z-score was >3.

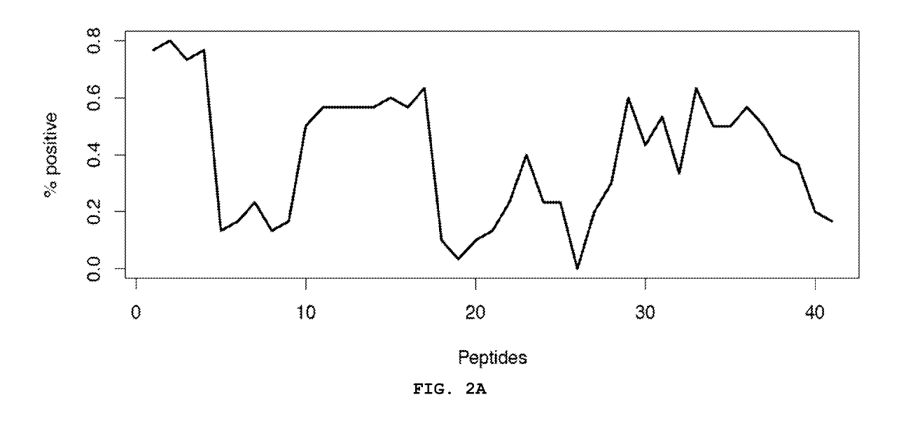

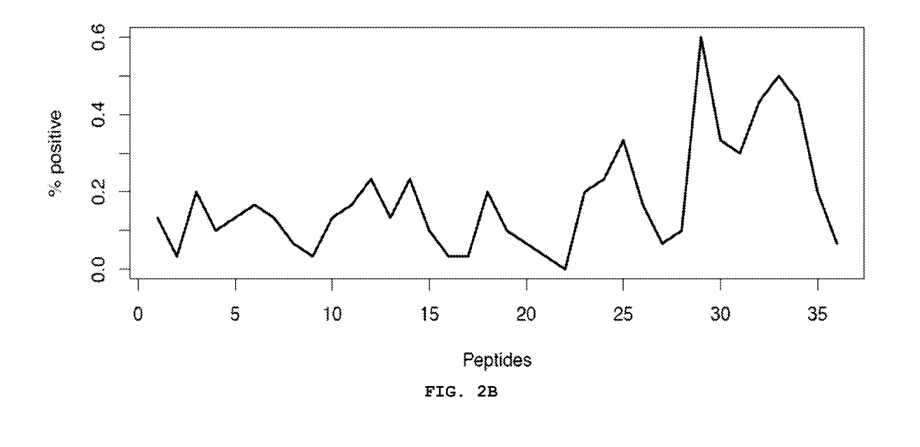

Epitopes. An IgE-binding epitope was defined as a region containing the same 6-13 amino acid sequence that was present in 3-5 contiguous peptides for which the mean z-score was >3 (FIGS. 1 and 2A-2B), similar to the approach described by Ayuso et al. in J Allergy Clin Immunol, 2010, 125, 1286-93 e3.

Immunodominance. IgE binding epitopes recognized by at least 50% of peanut-allergic subjects were considered immunodominant, similar to criteria previously published. See, for example, Shreffler et al., J Allergy Clin Immunol, 2004, 113, 776-82; Burks et al., J Clin Invest, 1995, 96, 1715-21; and Clarke et al., Clin Exp Allergy, 1998, 28, 1251-7.

TABLE A
Demographics, clinical history, and grade of reaction

| | | | | Age | Total IgE | Pnt IgE | | | | | | | | Rxn |
|---|---|---|---|---|---|---|---|---|---|---|---|---|---|---|
| Subject | Gender | Age | Race | onset | (ng/ml) | (ng/ml) | Hives | Rash | AE | UR | RESP | GI | êBP | Grade |
| D19 | M | 29 | WHITE | 11 M | 136 | 59 | Y | N | N | N | Y | N | N | 2 |
| D44 | M | 14 | WHITE | 1 Y 6 M | 1015 | 65.3 | N | N | Y | Y | Y | Y | N | 3 |
| D48 | M | 18 | WHITE | UNK | 138 | 71.1 | Y | Y | Y | N | Y | N | N | 2 |
| D50 | M | 11 | HISPANIC | 1 Y 1 M | 538 | 37.8 | Y | Y | Y | N | Y | Y | N | 3 |
| D53 | M | 11 | WHITE | 2 Y | 1185 | 74.7 | Y | N | Y | N | Y | Y | N | 3 |
| D59 | M | 10 | WHITE | 3 Y | 290 | 13.4 | Y | N | Y | N | Y | Y | N | 3 |
| D60 | M | 16 | WHITE | 3 Y | 454 | 159 | Y | Y | Y | N | Y | N | Y | 4 |
| D61 | F | 7 | WHITE | 2 Y | 2439 | 848 | Y | Y | Y | N | N | N | N | 1 |
| D63 | F | 19 | ASIAN | 5 Y | 3263 | 78.6 | Y | Y | Y | Y | Y | Y | N | 3 |
| D64 | F | 9 | WHITE | 7 M | 2803 | 591 | Y | Y | Y | N | Y | N | N | 2 |
| D65 | F | 11 | WHITE | 1 Y 6 M | 2421 | 180 | N | N | Y | Y | Y | N | N | 3 |
| D66 | M | 7 | WHITE | 1 Y | 186 | 77 | N | N | N | N | Y | Y | N | 3 |
| D67 | M | 9 | WHITE | 2 Y 4 M | 568 | 137 | Y | N | Y | N | Y | Y | N | 3 |
| D68 | F | 16 | WHITE | 3 Y | 212 | 25.2 | Y | Y | Y | N | Y | Y | Y | 4 |
| D68 | M | 12 | WHITE | 1 Y 1 M | 212 | 22.3 | Y | Y | Y | N | Y | N | Y | 4 |
| D70 | M | 13 | WHITE | 1 Y 4 M | 1423 | 500 | Y | N | Y | N | Y | N | N | 2 |
| D70B | M | 7 | WHITE | 1 Y | 245 | 52.7 | Y | Y | Y | N | Y | Y | Y | 4 |
| D71 | M | 7 | ASIAN/AF.AM | 2 Y 6 M | 4939 | 88.4 | Y | N | N | N | N | Y | N | 2 |
| D72 | M | 10 | WHITE | 1 Y 4 M | 474 | 14.1 | Y | Y | Y | N | N | N | N | 1 |
| D74 | F | 15 | HISPANIC | 2 Y | 194 | 28.9 | Y | Y | Y | N | Y | N | N | 3 |
| D77 | M | 11 | WHITE | 4 M | 505 | 48.7 | Y | Y | N | Y | Y | Y | N | 3 |
| D78 | M | 18 | WHITE | 16 Y | 1325 | 62.6 | N | Y | Y | N | N | N | N | 2 |
| D80 | M | 13 | WHITE | 2 Y 6 M | 545 | 164 | N | N | N | N | N | Y | N | 2 |
| D81 | M | 16 | WHITE | 2 Y | 268 | 67.5 | Y | Y | Y | N | Y | Y | N | 3 |
| D82 | F | 18 | WHITE | 3 Y 6 M | 265 | 18.9 | Y | Y | Y | Y | N | N | N | 3 |
| D98 | F | 26 | WHITE | 4 Y | 80.5 | 16.1 | Y | Y | Y | Y | Y | Y | Y | 4 |
| D103 | F | 9 | WHITE | 1 Y 6 M | 1797 | 787 | Y | Y | Y | N | Y | Y | N | 3 |
| D105 | M | 16 | WHITE | 1 Y | 88.2 | 14 | Y | Y | Y | N | N | Y | N | 2 |
| D207 | F | 32 | HISPANIC | 1 Y | 199 | 65.3 | Y | Y | Y | N | Y | Y | N | 3 |
| D114 | F | 28 | WHITE | 1 Y | 117 | 22.4 | Y | Y | Y | N | N | N | N | 1 |

RBL SX-38 cell assay, CPE and CPE depleted of Ara h 2 and Ara h 6 (CPE w/o 20 kD): RBL SX-38 cells were grown, sensitized and triggered with either crude peanut extract (CPE) or CPE depleted (>99%) of both Ara h 2 protein moiety and Ara h 6 protein moiety by removal of an ˜20 kD peak using gel filtration chromatography (CPE w/o 20 kD) as previously described. See, for example, Porterfield et al., Clin Exp Allergy, 2009, 39, 1099-108; and Kulis et al., Clinical and Experimental Allergy, 2011, In press, 2012. The effective concentration giving 50% of the maximal activity (EC₅₀) for each serum was determined from best fit plots and the percent contribution of Ara h 2 protein moiety and Ara h 6 protein moiety to the allergic effector activity of the CPE was calculated. For samples with ≦10-fold increase in EC₅₀, % loss of activity=(log₁₀ of the fold change)×90%). For samples with 10.1-100 fold increase in EC₅₀, % loss of activity=(90%+10%×(log₁₀ of the (fold change divided by 10)), for example, if there is a 50-fold change in the EC₅₀ with removal of Ara h 2 protein moiety and Ara h 6 protein moiety, the percent contribution is calculated as 90%+10% of the log₁₀ of 5)=90%+6.99%=96.99%. Those sera not assayed did not give sufficient degranulation for accurate determination of the EC₅₀ with CPE, were overly toxic to the cells, or were not available in sufficient quantity.

### Statistical Analyses

Cluster analysis. For the microarray data, heat maps and unsupervised hierarchical cluster analyses were performed with R-2.14.1 software for Windows (http://cran.r-project.org/bin/windows/base/) in order to see if patients sort into clusters based on clinical history. For analysis of z-scores, the entire range of z-scores (−2 to 28) was used but, to show the fine detail of binding at lower z-scores, the color scale in the figures (i.e., optically analyzable patterns) were adjusted to show values from −2 to ≧5. Additional analyses were based on binary binding (yes, z>3 or no, z≦3) to individual peptides and binary binding (yes, z>3 or no, z≦3) to epitopes.

GraphPad Prism 5.0c software for the Macintosh (GraphPad; La Jolla, Calif.) was used to generate graphs, to calculate EC₅₀ values and for statistical analysis. The following statistical tests were used: Spearman rank order correlation coefficients for correlations; a one sample Student's t test to evaluate the effect of removing Ara h 2/6 from CPE on the EC₅₀; Mann-Whitney test for comparisons of the number of binding events; and Fisher's exact test for comparing frequencies of two possible outcomes. All comparisons were two-tailed. P-values of less than 0.05 were considered to be significant.

### Results

Clinical information: Sera from 30 peanut allergic individuals were assessed. Reported symptoms were classified into grades of anaphylaxis (Table A). Three subjects (10%) were classified as grade I, 8 (27%) as grade II, 14 (47%) as grade III, and 5 (17%) as grade IV. Demographic and serologic data are shown in Table A. The total IgE, peanut specific IgE, and % peanut/total IgE had no discernable relationship to the severity of these reported clinical reactions to peanuts.

TABLE B
EC₅₀ with and without 20 kD fraction

| | | | Fold Δ in EC₅₀ due | % Allergic |
|---|---|---|---|---|
| | EC₅₀ (CPE) | EC₅₀ (ng/ml) | to removal of the | Effector |
| Serum | (ng/ml) | (CPE - 20 kD)* | 20 kD fraction | Activity^{‡} |
| D19 | 5.5 | 30.8 | 5.6 | 67 |
| D44 | 2.6 | 28 | 10.8 | 90 |
| D4S | 2.4 | 10.7 | 4.5 | 58 |
| D50 | 4.2 | 102 | 24.3 | 94 |
| D59 | 10.4 | 257 | 24.7 | 94 |
| D60 | 5.9 | >300 | 50.8 | 97 |
| D63 | 6.6 | 71 | 10.8 | 90 |
| D68 | 4.7 | 32.4 | 6.9 | 75 |
| D69 | 11 | 40.3 | 3.7 | 51 |
| D70 | 5.3 | 21.3 | 4.0 | 54 |
| D72 | 29.8 | >300 | 10.1 | 90 |
| D77 | 28.5 | 210.7 | 7.4 | 78 |
| D81 | 8.2 | >300 | 36.6 | 96 |
| D103 | 0.73 | 3.74 | 5.1 | 64 |
| D107 | 8.95 | 48.3 | 5.4 | 66 |
| Mean | 9.0 | 117 | 14 | 78 |
| SEM | 2.2 | 31 | 3.6 | 4.2 |

*The EC50 was greater than the highest concentration of stimulus tested (300 ng/ml) so this value was used as a conservative estimate.
^{‡}In the 20 kD fraction.

Functional assay: Mediator release assays were performed with IgE from 15 of the 30 subjects. For all 15 sera examined, the EC₅₀ was shifted by 14±4 fold (mean±SEM; 95% confidence interval, 6.3 to 22 fold; p<0.002 compared with no effect) by removing the 20 kD fraction containing Ara h 2 and 6 and demonstrating that these two allergens together account for 78±4% (mean±SEM; 95% confidence interval, 70% to 86%) of the total effector activity in the CPE (Table B) (p<0.0001 compared with no effect). Individual RBL assay results for the 15 subjects (D19; D44; D48; D50; D59; D60; D61; D63, D68; D70; D72; D77; D81; D103, and D107) assayed were also graphed (not shown) comparing assay results of CPE and CPE w/o 20 kD. Data were normalized to “net” degranulation±SD (n=3 wells/observation) after subtraction of background (9±1%, mean SEM; n=15 experiments). These results confirm the present inventors' previous findings with serum pools and small numbers of sera that Ara h 2 and Ara h 6 are crucial effector molecules. Of note, there does not appear to be any suggestion of a relationship between the percent contribution of Ara h 2 and Ara h 6 to the total effector activity and severity of reported reactions.

IgE binding to linear peptides: The percent of 30 sera that contained IgE binding to each of the peptides of Ara h 2 and Ara h 6 is shown in FIGS. 2A and 2B, respectively. Similar to previous reports, there was binding to broad range of Ara h 2 peptides with frequent binding to peptides: 1-5, 11-18, 22-23 and 28-40 (FIGS. 1 and 2A). Surprisingly and unexpectedly, the number of subjects with IgE that bind peptides of Ara h 6 was more limited, with the C-terminus (peptides 29-34) being most frequently recognized (FIGS. 1 and 2B). Furthermore, there was no significant correlation between the intensity of the signal and the severity of the reported clinical reactions (data not shown).

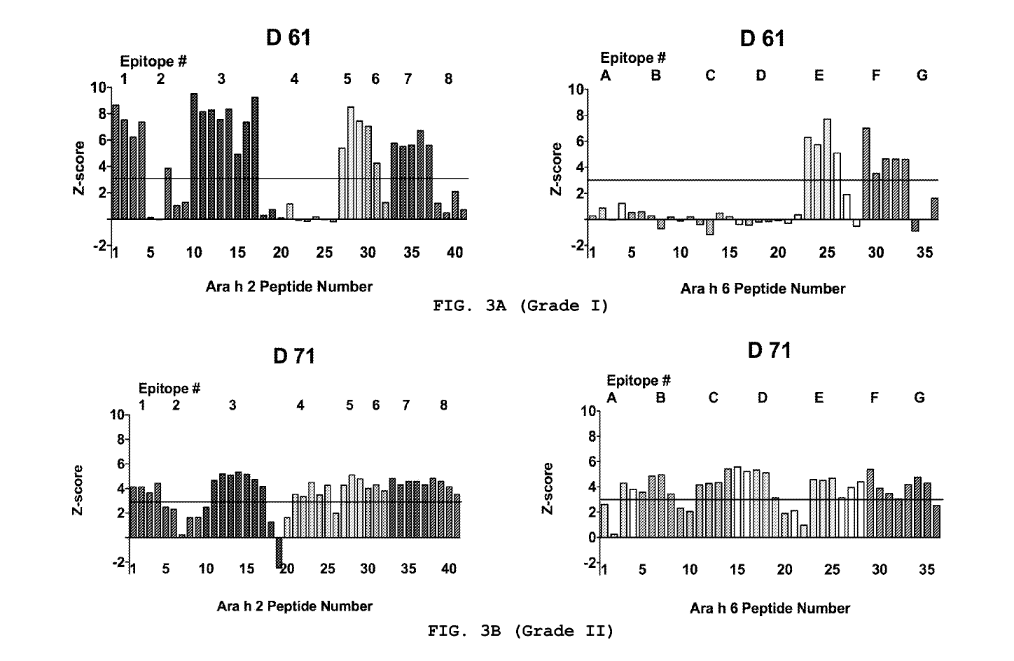

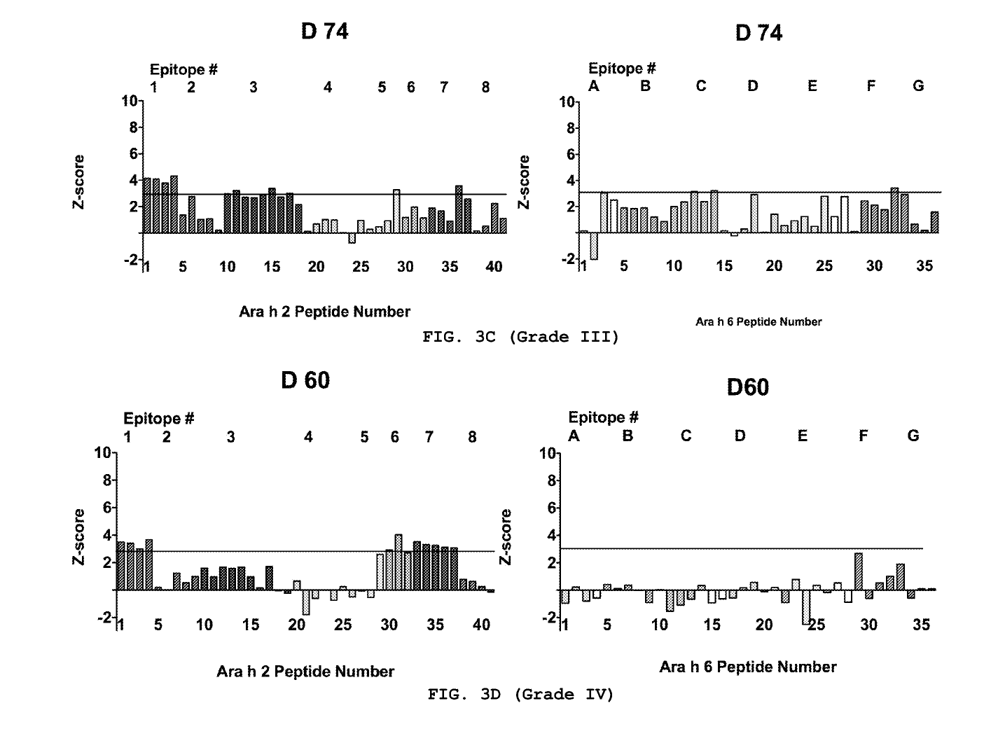

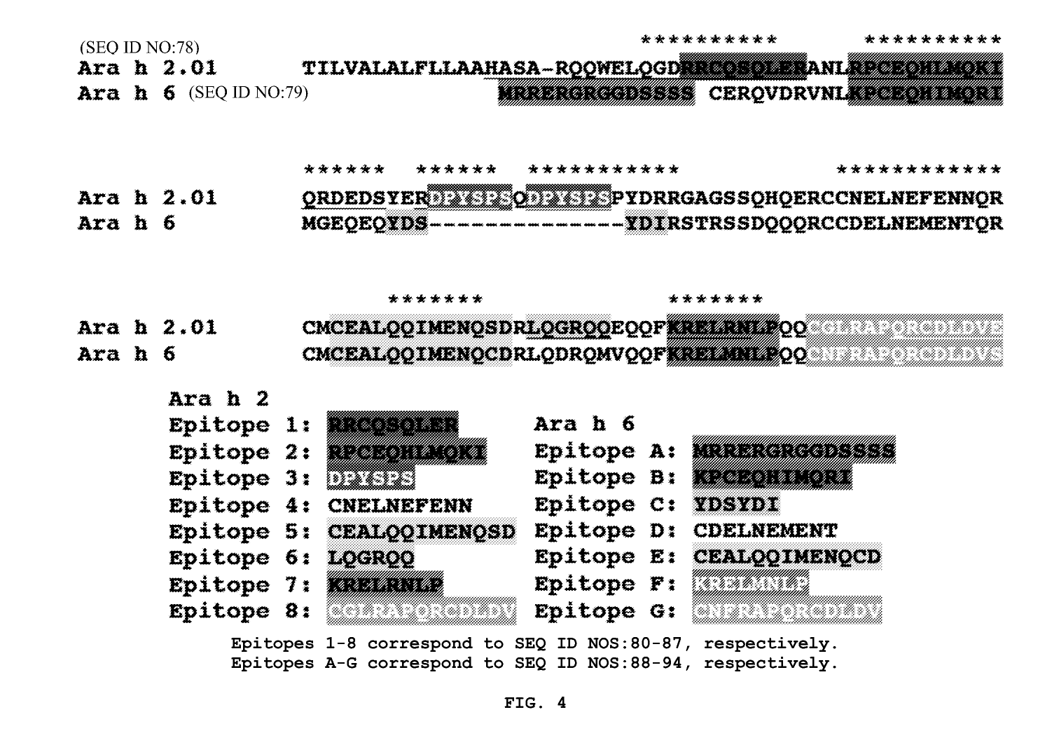

Identification of IgE binding fingerprints to peptides of Ara h 2 and Ara h 6 for each individual: Data for binding of sera from each of the 30 individuals to the individual peptides for both Ara h 2 and Ara h 6 were obtained. A representative IgE binding and peanut allergy grade are shown in FIGS. 3A-3D. Positive binding (i.e., binding above the threshold value) was defined as z-score greater than 3 shown as a horizontal line on each graph. Each subject revealed a distinctive IgE binding configuration, or fingerprint, which remained consistent with different concentrations of serum and after normalizing the data based on the intensity of binding to either Ara h 2 and/or Ara h 6 within each array (data not shown). Moreover, these individual peptide-binding fingerprints, when coalesced into epitope-binding, exhibited characteristic patterns when the subjects were stratified according to historical clinical reactions to peanuts.

Ara h 2 and Ara h 6 share homologous epitopes: Epitope mapping was performed to see if binding to Ara h 2 and Ara h 6 reflected the known homology between these proteins as well as to remove the redundancy that may arise by virtue of overlapping peptides. Eight IgE-binding epitopes were identified for Ara h 2, consistent with published data. The epitopes of Ara h 2 are notated in this report as epitopes 1-8 (FIG. 4, SEQ ID NOS:80-87, respectively). Seven novel IgE-binding epitopes were identified for Ara h 6 and, to distinguish these from those of Ara h 2, they are notated as epitopes A-G (FIG. 4, SEQ ID NOS:88-94, respectively). Two epitopes of Ara h 6 (A and C) are unique with no homology (0%) to Ara h 2 whereas 5 epitopes (B, D, E, F, and G) are highly homologous (70-93%) with epitopes of Ara h 2 (2, 4, 5, 7, and 8 respectively). Epitope 6 in Ara h 2 shares homology to a corresponding sequence in Ara h 6 (67%). However, due to lack of significant IgE binding (mean z score<3) this region in Ara h 6 was not identified as an IgE-binding epitope. The color-coding and/or underlining of the epitopes in FIGS. 1 and 4 corresponds to that of the bars in FIGS. 3A-3D.

As expected, the significance tests of the Spearman rank correlation coefficients indicated strong correlations between IgE binding (z-scores) to Ara h 2 epitope 2 and Ara h 6 epitope B (homology=81%; r=0.55, p=0.001), Ara h 2 epitope 4 and Ara h 6 epitope D (homology=70%; r=0.64, P<0.001), Ara h 2 epitope 5 and Ara h 6 epitope E (homology=92%; r=0.65, p<0.001), Ara h 2 epitope 7 and Ara h 6 epitope F (homology=83%; r=0.82, p<0.0001), and Ara h 2 epitope 8 and Ara h 6 epitope G (homology=85%; r=0.90, p<0.0001) (data not shown). There was modest correlation for Ara h 2 epitope 1 and Ara h 6 epitope A (homology=0%; r=0.37, p=0.04) in spite of no homology in the linear sequence perhaps due to the fact that these epitopes are located on similar surface positions. There was no significant correlation between binding for the non-homologous Ara h 2 epitope 3 and Ara h 6 epitope C (homology=0%; r=0.21, p=not significant (ns)).

IgE binds to more epitopes of Ara h 2 than of Ara h 6: Regardless of clinical history, IgE from subjects bound much more frequently (z>3) to epitopes to epitopes of Ara h 2 (median=3 epitopes/serum (E/S)) (Table C) compared with binding of IgE to the Ara h 6 epitopes (Table D) (median=1 E/S; p=0.0002). In that 30 sera that could potentially bind to each of 8 epitopes of Ara h 2 and to 7 epitopes of Ara h 6, it was determined that there were 240 and 210 potential binding events (PBE) for Ara h 2 and Ara h 6, respectively. For all 30 sera, there were 100 binding events (BE) for Ara h 2 (Table C) compared to 40 for Ara h 6 (Table D) (p<0.0001). For Ara h 2, epitopes 1 (z>3 for 23 sera), 3 (z>3 for 18 sera) and 7 (z>3 for 17 sera) are immunodominant. For Ara h 6, only epitope F (z>3 for 15 sera) met the criteria for immunodominance.

Characteristic patterns of IgE-binding to epitopes of Ara h 2 and Ara h 6 emerge when subjects are stratified according to clinical histories: Characteristic patterns emerged when IgE-binding to epitopes for an individual was examined in the context of their historical reactions to peanut exposure. For Ara h 2 (Table C), 52 BE out of 152 PBE (34%) for the 19 subjects with more severe histories (grades III+IV) were found compared to 48 BE out of 88 PBE (55%) for the 11 subjects with less severe histories (grades I+II) (p=0.0027). For Ara h 6 (Table D), only 16 BE out of 133 PBE (12%) for the 19 subjects with grades

TABLE D

| | | Epitope | | | | | | |
|---|---|---|---|---|---|---|---|---|
| Symptoms | Serum | A | B | C | D | E | F | G |
| Grade I | D61 | | | | | Y | Y | |
| | D72 | | | | | | | |
| | D114 | | | | | Y | | |
| Grade II | D48 | | | Y | | Y | Y | Y |
| | D64 | | Y | | | Y | Y | Y |
| | D70 | | | | | | Y | |
| | D71 | | Y | Y | Y | Y | Y | Y |
| | D78 | Y | | | | | | |
| | D80 | Y | | Y | | | Y | |
| | D105 | | | | | | | |
| | D19 | | | | | | Y | Y |
| Grade III | D44 | Y | | | Y | Y | Y | |
| | D50 | | | | | | Y | |
| | D53 | | | | | | | |
| | D59 | | | | | | | |
| | D63 | | | | | | | |
| | D65 | | | | | | | |
| | D66 | | | Y | | | | |
| | D67 | | | | | | | |
| | D74 | | | | | | | |
| | D77 | | | | | | | |
| | D81 | | | | | | Y | |
| | D103 | | | | | | | |
| | D107 | | | | | Y | Y | |
| | D82 | | | | | | Y | |
| Grade IV | D60 | | | | | | | |

TABLE C
Binary binding of IgE to epitopes of Ara h 2

| | | Epitope | | | | | | |
|---|---|---|---|---|---|---|---|---|
| Symptoms | Serum | 1 | 2 | 3 | 4 | 5 | 6 | 7 |
| Grade I | D61 | Y | | Y | | Y | Y | Y |
| | D72 | | | | | | | |
| | D114 | Y | Y | | Y | Y | | |
| Grade II | D48 | Y | | Y | Y | Y | Y | Y |
| | D64 | | Y | Y | | Y | Y | Y |
| | D70 | Y | | Y | | | | Y |
| | D71 | Y | | Y | Y | Y | Y | Y |
| | D78 | Y | | Y | | | Y | Y |
| | D80 | | | Y | | | Y | Y |
| | D105 | Y | | | Y | Y | | |
| | D19 | Y | | | Y | Y | | Y |
| Grade III | D44 | Y | | Y | Y | | Y | Y |
| | D50 | | | | | | | Y |
| | D53 | | | | | | | |
| | D59 | | | | | | | |
| | D63 | | | Y | | | | |
| | D65 | Y | | | | | Y | |
| | D66 | Y | | Y | | | | |
| | D67 | Y | | Y | | | | Y |
| | D74 | Y | | | | | | |
| | D77 | Y | | Y | | | | |
| | D81 | Y | Y | Y | | | | Y |
| | D103 | Y | | Y | | | Y | |
| | D107 | Y | | Y | | Y | Y | Y |
| | D82 | Y | Y | Y | | | | Y |
| Grade IV | D60 | Y | | | | | Y | Y |

III+IV were found compared to 24 BE out of 77 PBE (31%) for the 11 subjects with less grades I+II (p=0.001). Thus, IgE from patients with more severe clinical histories recognize fewer linear epitopes of both Ara h 2 and Ara h 6.

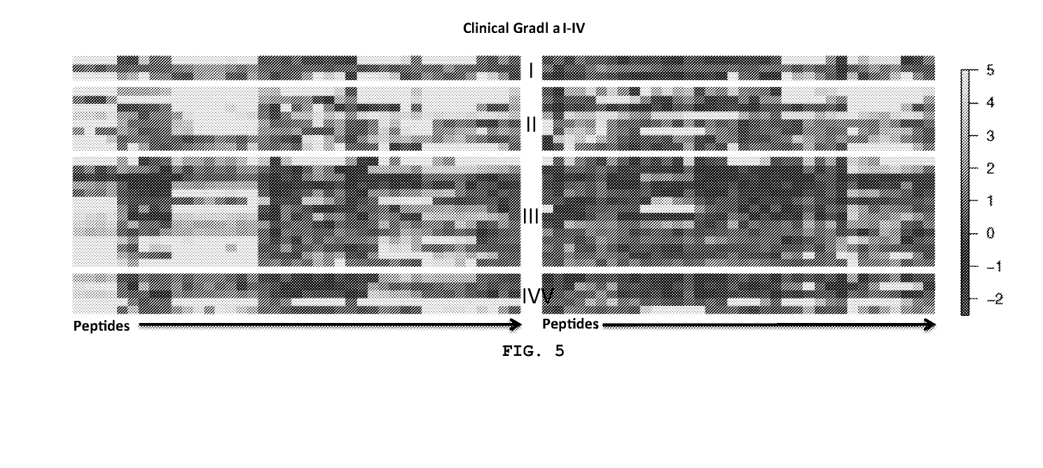

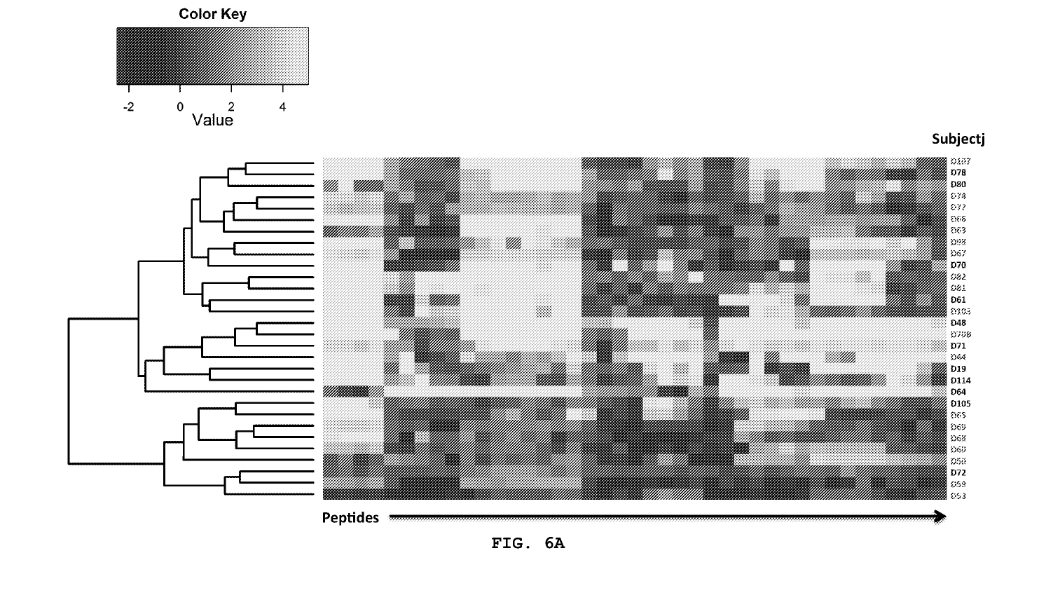

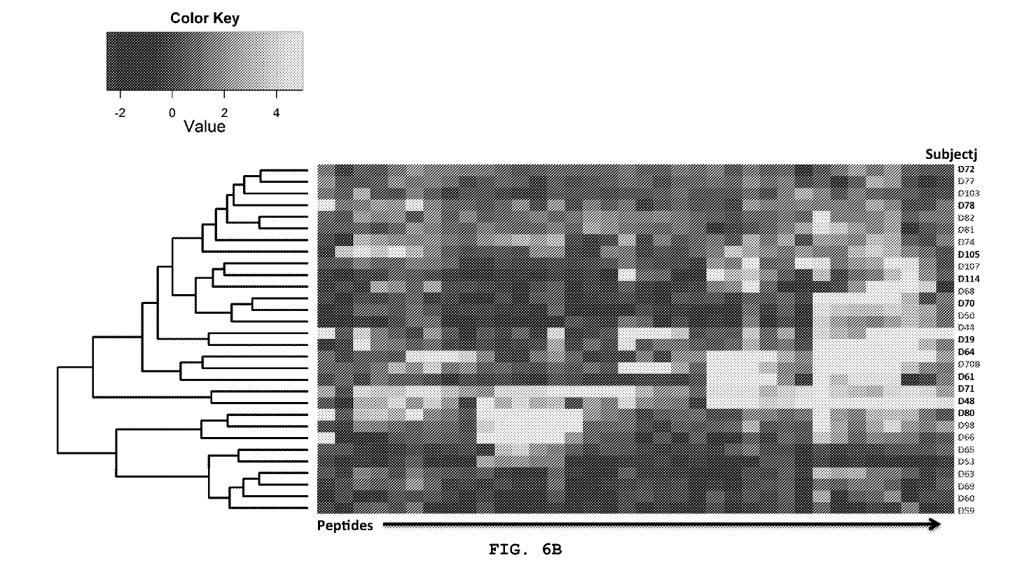

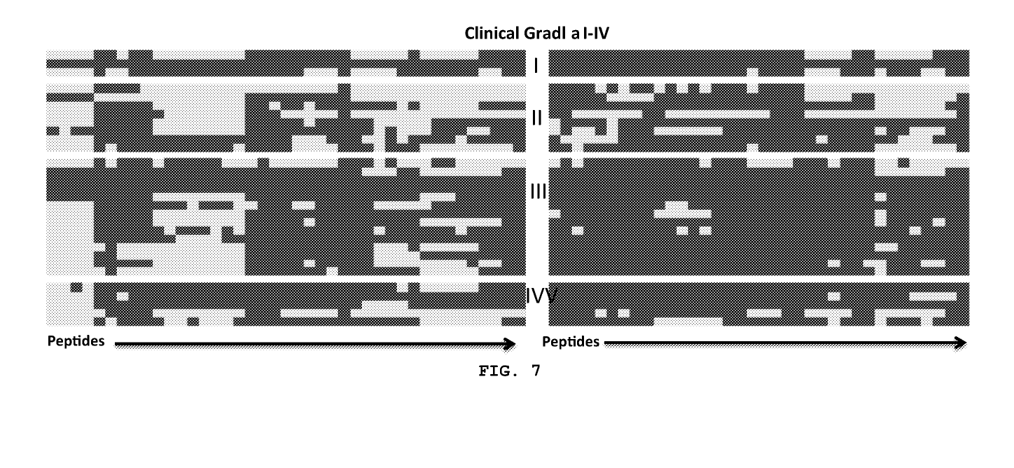

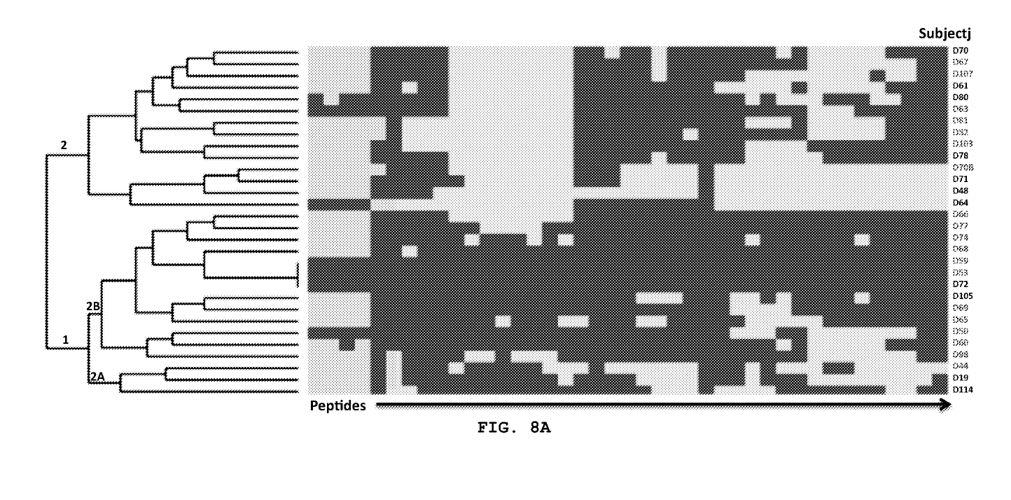

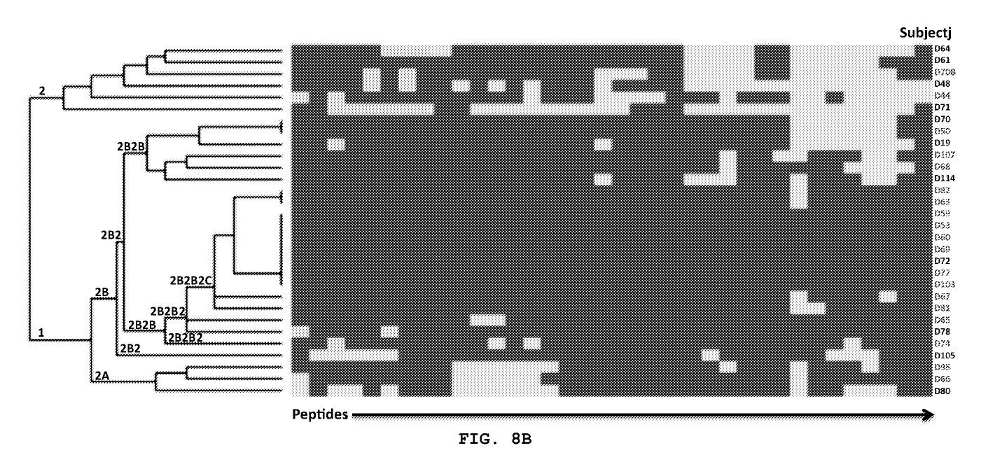

Hierarchal analyses of IgE binding to peptides: A group-peptide plot (FIG. 5) was generated and an unsupervised hierarchal cluster analysis was performed for the 30 subjects using the intensity of IgE binding (z-scores) to each peptide (FIGS. 6A and 6B). Patients with more severe clinical histories (shown in red numbers) did not cluster significantly into specific groups. Therefore, data was reexamined based on positive or negative binding (binary; yes, z>3 or no, z≦3) to the peptides (FIGS. 7 and 8). In the unsupervised cluster analysis (FIG. 8), it was found that 11 out of 13 patients who clustered into node 1B of Ara h 2 had grades III/IV severity compared to 8 subjects of the 17 remaining patients who did not cluster into this node (p=0.058, ns) (FIG. 8A). On the other hand, 12 out of 14 subjects who clustered into node 1B2A of Ara h 6 had grades III/IV severity compared to 7 individuals out of 16 remaining subjects who did not cluster into this node (p=0.026) (FIG. 8B).

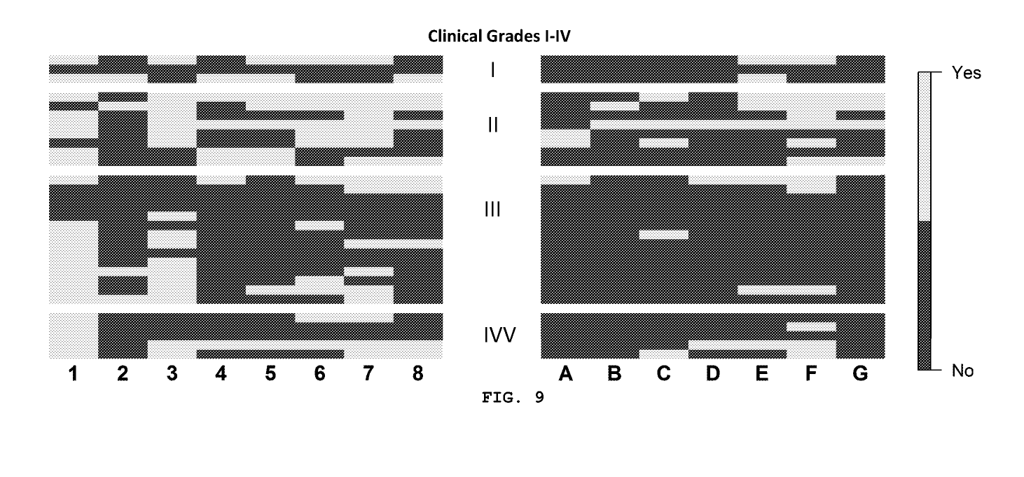

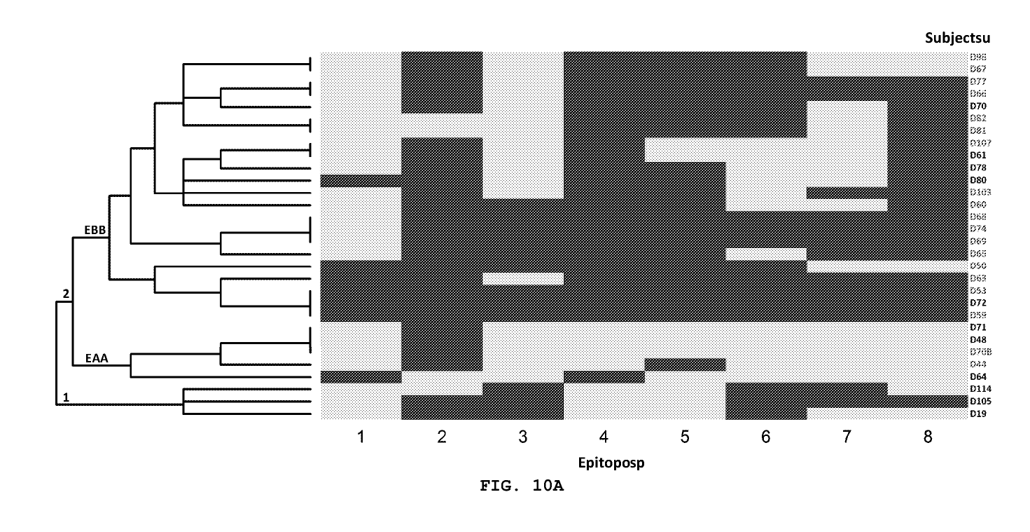

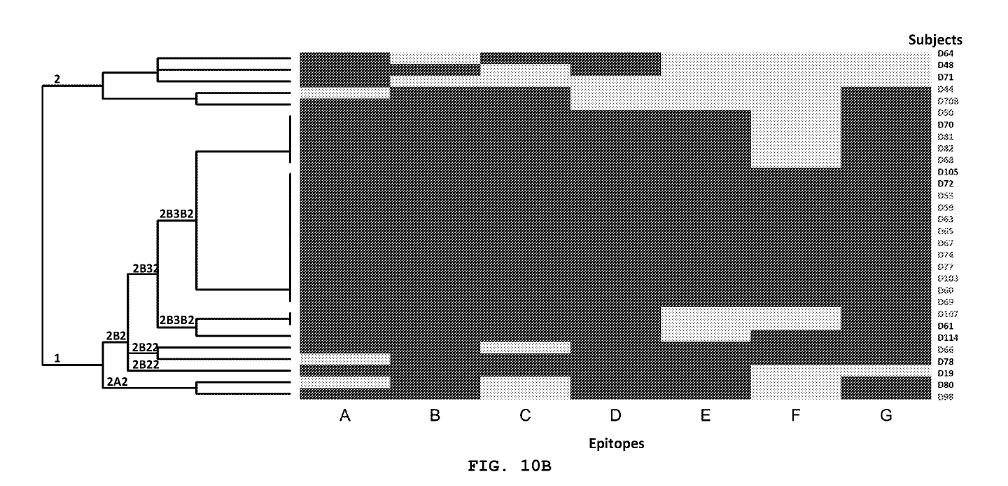

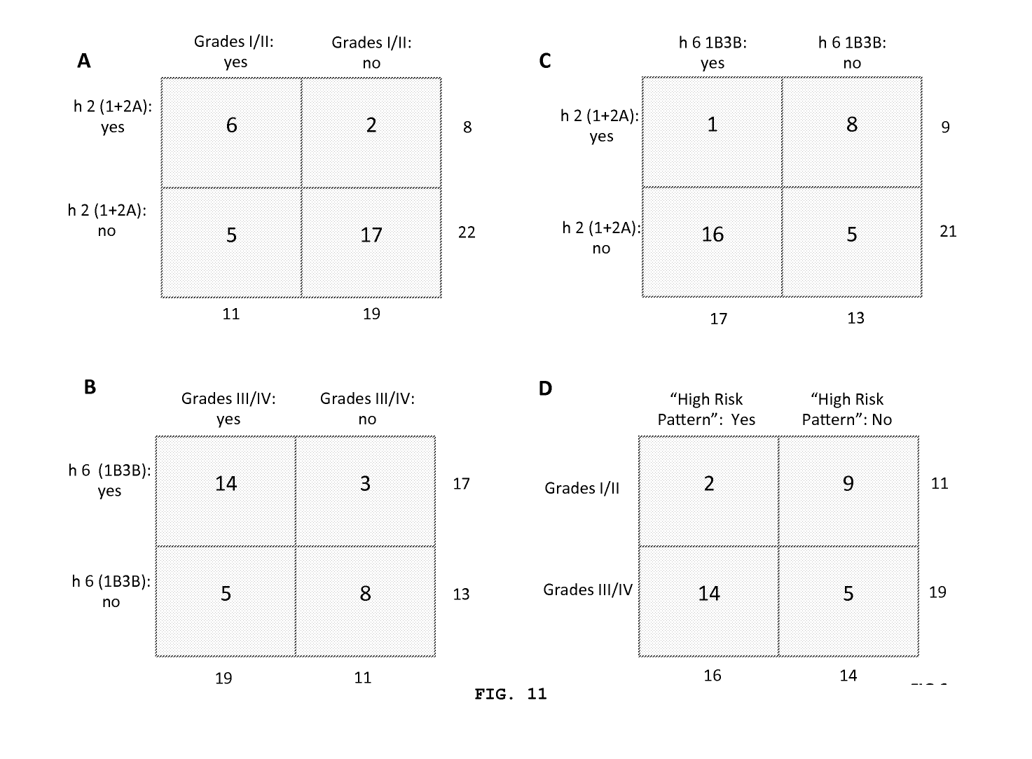

Hierarchal analyses of IgE binding to epitopes: Data was examined based on positive or negative binding to epitopes rather than to peptides, and a group-epitope heat map was generated (FIG. 9). An unsupervised hierarchical cluster analysis was also performed (FIGS. 10A and 10B). For Ara h 2 (FIG. 10A), 6 out of 8 subjects who clustered into node 1 and node 2A together have a history of grade I/II severity compared to 5 individuals with grades I/II severity out of 22 remaining subjects who did not cluster to this node (FIG. 11A; p=0.028). This indicates that individuals who cluster into this node are likely to have milder symptoms (grades I/II). For Ara h 6 (FIG. 10B), the largest single node, 1B3B, contains 17 subjects. Fourteen out of 17 subjects in this node have a history of grade III/IV severity compared to 5 individuals with grades III/IV out of the 13 remaining subjects who did not cluster into this node (FIG. 11B; p=0.023). Thus, individuals clustering into this node are likely to demonstrate more severe symptoms (grades III/IV).

Moreover, out of the 17 individuals who clustered to Ara h 6 1B3B, only one subject clustered to either Ara h 2 node 1 or 2A (FIG. 11C). This illustrates that subjects who cluster into node 1B3B of Ara h 6, which is populated predominantly with individuals who have a history of grades III/IV severity, do not cluster into nodes (1+2A) of Ara h 2, which is populated predominantly with those who have a history of grades I/II severity. Thus, a “high-risk pattern” is identified in which subjects with high grades of severity cluster to node IB3B of Ara h 6 but not to nodes (1+2A) of Ara h 2. As shown in FIG. 11D, 16 subjects have this high-risk pattern, and of these 14 have a history of grades III/IV severity compared to only two with a history of grades I/II severity. Moreover, out of the 14 subjects who did not demonstrate this high-risk pattern, 9 individuals had grades I/II severity (OR=12.6; 95% CI=2.0-79.5; p=0.0068).

### Discussion

As illustrated herein, threshold values of binding and optical patterns generated therefrom using peptide microarrays can be used to examine IgE binding to linear peptides and to epitopes of the highly potent peanut allergens, Ara h 2 and Ara h 6 for 30 subjects with strong histories and serologic evidence of clinically important peanut allergy. Based on clinical history from a detailed questionnaire obtained at enrollment, the severity of allergic reaction were classified based on the reported naturally occurring reactions to ingestion of peanuts. In this particular example, the severity of allergic reaction were based on degrees of anaphylaxis. One can use historical clinical data or data obtained via double-blind placebo-controlled food challenge as long as the classification is internally consistent. As illustrated herein, methods of the invention have revealed dramatic findings that have a wide variety of significant utilities.

In another aspect of the invention and in contrast to conventional methods for testing peanut allergy, the present inventors have discovered that Ara h 2 and Ara h 6 are crucial effector molecules. As illustrated herein, the present inventors have discovered the contribution of Ara h 2/6 to the effector function for 15 of the 30 sera. Data provided herein demonstrate that Ara h 2/6 account for 78±4% of the allergic effector of the CPE (n=15; p<0.0002) (Table B).

The present inventors have also determined IgE-binding to linear peptides of Ara h 2 and Ara h 6. Unlike previous reports where all sera were diluted similarly (e.g., 1:5) despite variations in peanut-specific IgE, methods of the invention include normalizing the fluid sample. For example, in the examples illustrated herein all the serum were normalized such that the concentration of IgE was about 22 ng/ml, thereby removing a bias toward sera containing high levels of peanut-specific IgE. In a secondary analysis, data were further normalized based on binding of IgE to native Ara h 2 and/or Ara h 6 within each microarray assay. The latter normalization may not be necessary in some instances, as this normalization did not significantly change the final data (data not shown).

As illustrated herein, using a threshold binding value (e.g., via microarray analysis) between an immunoglobulin and marker proteins provides a significantly more accurate determination of a subject's degree of severity of allergic reaction. For example, as shown in the above example, using distinct IgE-binding fingerprints (e.g., reference patterns produced using threshold binding values) to peptides for Ara h 2 and Ara h 6 for each serum assayed (see, for example, FIGS. 3A-3D), methods of the invention allow a significantly more accurate determination of severity of a subject's peanut allergy. These individual IgE-binding fingerprints to peptides taken alone were unrevealing, but when they were coalesced based on epitope-binding and sorted according to clinical history, distinctive patterns emerged. The epitopes identified for Ara h 2 were similar to those previously reported. See, for example, Shreffler et al., J Allergy Clin Immunol, 2005, 116, 893-9 and Stanley et al., Arch Biochem Biophys, 1997, 342, 244-53. Eight IgE-binding epitopes were identified for Ara h 2, and 7 for Ara h 6 (FIG. 4). Six regions showed positive correlations between homologous epitopes of Ara h 2 and Ara h 6 (p<0.001) (data not shown).

Overall, individual IgE samples bound linear epitopes of Ara h 2 more frequently than epitopes of Ara h 6 (p<0.002) (Table C). Surprisingly and unexpectedly, IgE from subjects with more severe histories of allergic responses to peanut exposure recognized a significantly more limited breadth of linear epitopes compared to those who reported less severe responses to peanuts (p<0.001). This result is in contrast to what has been described in literature to date. See, for example, Shreffler et al., J Allergy Clin Immunol, 2004, 113, 776-82; Flinterman et al., J Allergy Clin Immunol, 2008, 121, 737-43 e10; and Shreffler et al., J Allergy Clin Immunol, 2005, 116, 893-9. It is believed that this difference results from the fact that previous studies did not adjust the sera to the same anti-peanut IgE concentrations (i.e., no normalization) and focused on the degree of binding to peptides rather than threshold binding values, e.g., binary (yes/no) binding to epitopes.

As illustrated in the example above, in an unsupervised hierarchal cluster analysis, a “high-risk pattern” was identified in which patients with high grades of severity cluster to node IB3B of Ara h 6 but not to nodes (1+2A) of Ara h 2 (FIG. 11D). Sixteen individuals have this high-risk pattern, and of these 14 have a history of grades III/IV severity compared to only two subjects who have a history of grades I/II severity. Of note, node 1B3B of Ara h 6 is characterized by either binding only to epitope F, or by no detectable binding above predetermined threshold (z>3) to any IgE-binding linear epitopes of Ara h 6 (FIG. 10B). Thus, peanut allergic patients with more severe reactions appear to have more IgE antibody that recognizes conformational rather than linear epitopes.

Subjects with histories of more severe clinical reactions have characteristic patterns of binding to linear epitopes of Ara h 2 and Ara h 6 and this have a wide variety of utilities, for example, in predicting clinical reactions from future exposure to peanuts, screening and/or identification of compounds for treating or alleviating peanut allergies. The overall approach to focus on IgE-binding epitopes of the most potent allergens identified in functional assays is also be useful for other IgE-mediated food allergies.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for determining a likelihood of degree of severity of allergic reaction to an allergy inducing material in a subject, said method comprising:
contacting a fluid sample of the subject with a test composition comprising a marker protein moiety of the allergy inducing material, wherein the fluid sample comprises an immunoglobulin of the subject in which the immunoglobulin is known to cause an immune response in the presence of the marker protein in a control group that is allergic to the allergy inducing material, and wherein the marker protein moiety comprises a peptide moiety that is known to cause an immune response in the control group that is allergic to the allergy inducing material; and
analyzing the value of binding between the marker protein moiety of the allergy inducing material and the immunoglobulin in the fluid sample of the subject to determine the subject's likelihood of degree of severity of allergic reaction to the allergy inducing material.

2. The method of claim 1, wherein the test composition comprises a plurality of marker protein moieties of the allergy inducing material.

3. The method of claim 1, wherein fluid sample of the subject comprises blood, serum, plasma, and body secretions such as nasal secretions and fluid obtained from the lung (bronchoalveolar lavage fluid), or a combination thereof.

4. The method of claim 1, wherein the test composition comprises a microarray.

5. The method of claim 1, wherein said method of analyzing the value of binding between the marker protein moiety of the allergy inducing material and the immunoglobulin in the fluid sample of the subject comprises the steps of:
converting the value to an optically analyzable pattern; and
comparing the optically analyzable pattern to a reference pattern.

6. The method of claim 5, wherein the reference pattern comprises an optically analyzable pattern of threshold value of binding between the marker protein moiety of the allergy inducing material and the immunoglobulin in the fluid sample of the control subject that is allergic to the marker protein moiety of the allergy inducing material.

7. The method of claim 5, wherein the reference pattern comprises an optically analyzable pattern of binding threshold value between the marker protein moiety of the allergy inducing material and the immunoglobulin in the fluid sample of the control subject that is less allergic to the marker protein moiety of the allergy inducing material.

8. The method of claim 5, wherein the reference pattern comprises an optically analyzable pattern of threshold value of binding between the marker protein moiety of the allergy inducing material and the immunoglobulin in the fluid sample of the control subject that is substantially not allergic to the marker protein moiety of the allergy inducing material.

9. The method of claim 1 further comprising the step of normalizing the fluid sample concentration of the subject prior to said step of contacting the fluid sample of the subject with the test composition.

10. A method for predicting a likelihood of a degree of peanut allergy severity in a subject, said method comprising:
contacting a fluid sample of the subject comprising immunoglobulin E (IgE) with a test composition comprising a marker protein moieties of peanut, wherein the marker protein moiety comprises a mixture of: (i) Ara h 2 protein moiety; and (ii) Ara h 6 protein moiety; and
analyzing a value of binding between the marker protein of peanut and IgE in the fluid sample of the subject to determine the subject's likelihood of degree of severity of allergic reaction to peanut.

11. The method of claim 10, wherein fluid sample of the subject comprises blood, serum, plasma, and body secretions such as nasal secretions and fluid obtained from the lung (bronchoalveolar lavage fluid), or a combination thereof.

12. The method of claim 10, wherein the test composition comprises a microarray.

13. The method of claim 10, wherein said method of analyzing the binding value comprises the steps of:
converting the binding value to an optically analyzable pattern; and
comparing the optically analyzable pattern to a reference pattern.

14. The method of claim 13, wherein the reference pattern comprises an optically analyzable pattern of threshold value of binding between Ara h 2 protein moiety and Ara h 6 protein moiety of peanuts and IgE in the fluid sample of the control subject that is allergic to peanuts.

15. The method of claim 13, wherein the reference pattern comprises an optically analyzable pattern of threshold value of binding between Ara h 2 protein moiety and Ara h 6 protein moiety of peanuts and IgE in the fluid sample of the control subject that is substantially not allergic to peanuts.

16. The method of claim 10 further comprising the step of normalizing the fluid sample concentration of the subject prior to said step of contacting the fluid sample of the subject with the test composition.

17. The method of claim 10, wherein the marker protein moiety comprises a plurality of Ara h 2 protein moieties, a plurality of Ara h 6 protein moieties, or a combination thereof.

18. The method of claim 10, wherein the marker protein moiety is selected from the group consisting of SEQ ID NOS:1-77, SEQ ID NOS:80-94, or a combination thereof.

19. The method of claim 18, wherein the marker protein moiety comprises a plurality of a first oligopeptide moieties each of which is independently selected from the group consisting of SEQ ID NOS:1-41 and SEQ ID NOS:80-87 and a plurality of a second oligopeptide moieties each of which is independently selected from the group consisting of SEQ ID NOS:42-77 and SEQ ID NOS:88-94.

20. The method of claim 19, wherein the marker protein moiety comprises at least eight different first oligopeptides and at least seven different second oligopeptides.
